(19) United States
(12) Patent Application Publication
(10) Pub. No.: US 2026/0318924 A1
(43) Pub. Date: Oct. 15, 2026

(54) DISEASE-SPECIFIC BIOMARKER FOR PREDICTING AND DIAGNOSING PREECLAMPSIA AT EARLY PREGNANCY STAGE, AND USE THEREOF

(71) Applicant: SUNGKWANG MEDICAL FOUNDATION, Seoul (KR)

(72) Inventors: Hyun Mee RYU, Seongnam-si Gyeonggi-do (KR); Ji Hyae LIM, Seongnam-si Gyeonggi-do (KR)

(73) Assignee: SUNGKWANG MEDICAL FOUNDATION, Seoul (KR)

(21) Appl. No.: 19/687,765

(22) Filed: May 26, 2026

Related U.S. Application Data
(63) Continuation of application No. PCT/KR2024/016713, filed on Oct. 29, 2024.

(30) Foreign Application Priority Data
Nov. 27, 2023 (KR) 10-2023-0166684

(51) Int. Cl. G01N 33/68; C12Q 1/6883

(52) U.S. Cl. CPC G01N 33/6893; C12Q 1/6883; C12Q 2600/158; G01N 2800/368; G01N 2800/50

(57) ABSTRACT

The present invention relates to a disease-specific biomarker for early prediction and diagnosis of preeclampsia, and use thereof. A composition for predicting or diagnosing preeclampsia or a method for providing information for prediction or diagnosis of preeclampsia, according to one aspect, can simply and effectively predict or diagnose a disease by measuring and comparing the level of mRNA of a disease-specific protein that changes in a patient, or an mRNA of a gene encoding same.

### CROSS-REFERENCE TO RELATED APPLICATIONS

This application is a continuation of International Application No. PCT/KR2024/016713 filed on Oct. 29, 2024, which claims priority to Korean Patent Application No. 10-2023-0166684 filed on Nov. 27, 2023, the entire contents of which are herein incorporated by reference.

### TECHNICAL FIELD

The present disclosure relates to a disease-specific biomarker for early prediction and diagnosis of preeclampsia and its uses.

### BACKGROUND ART

Preeclampsia (PE), which is a condition characterized by hypertension (>140/90 mmHg) accompanied by proteinuria (>300 mg/day) caused by various factors such as inflammation, hypoxia, and oxidative stress after 20 weeks of gestation, is a significant cause of maternal mortality, occurring in 7-12% of pregnant women, and accounts for a large proportion of fetal and neonatal mortality and morbidity. If it progresses to a severe stage, it may lead to pulmonary edema, cerebral hemorrhage, liver and kidney failure, blood coagulation abnormalities, and even the death of the mother. Furthermore, impaired blood flow to the placenta and fetus results in fetal growth failure, which in severe cases may lead to fetal death. However, the exact etiology and treatment methods have not yet been clearly identified, and the only treatment is to end the pregnancy through delivery. Nevertheless, since the occurrence of preeclampsia may increase the fetal mortality rate due to premature birth, early diagnosis and prolonging delivery as much as possible may currently be the best feasible way to select.

Although diagnosis is available for pregnant women after 20 weeks of gestation (multiple cases after 32 weeks) using various indicators such as hypertension, proteinuria, and edema, there is no clearly established method for early diagnosis of preeclampsia to reduce fetal preterm birth induced by preeclampsia. Currently, as one of the diagnostic methods for preeclampsia, tests using plasma markers have been developed, but the PerkinElmer DELFIA® Xpress PIGF kit shows a false positive rate of 5% and a detection rate of 44%, while the Alere Triage® PIGF test lacks proven clinical diagnostic utility, resulting in continued low accuracy and insufficient clinical utility.

Accordingly, to prevent preeclampsia and improve prognosis through treatment at the onset of the disease, there is a need for a test method that may effectively predict preeclampsia in the early and middle stages of pregnancy and simplify diagnosis in the middle and late stages of pregnancy; however, there is currently no such technology among those developed to date.

Accordingly, the inventor(s) have discovered the world's first preeclampsia-specific biomarker, which exhibits significant changes in patients, and have derived a simple and effective method for identifying early prediction and diagnosis of preeclampsia.

### DISCLOSURE OF INVENTION

### Technical Goals

One aspect is to provide a biomarker composition for predicting or diagnosing early-, mid-, or late-pregnancy preeclampsia, including a protein or a gene encoding the protein.

Another aspect is to provide a use for predicting or diagnosing early-, mid-, or late-pregnancy preeclampsia, of a biomarker including a protein or a gene encoding the protein.

Another aspect is to provide a composition for predicting or diagnosing early-, mid-, or late-pregnancy preeclampsia, including a formulation for measuring a level of a protein or a level of mRNA of a gene encoding the protein.

Another aspect is to provide a use for predicting or diagnosing early-, mid-, or late-pregnancy preeclampsia, of a formulation for measuring a level of a protein or a level of mRNA of a gene encoding the protein.

Another aspect is to provide a use for producing a composition for predicting or diagnosing early-, mid-, or late-pregnancy preeclampsia, of a formulation for measuring a level of a protein or a level of mRNA of a gene encoding the protein.

Another aspect is to provide a kit for predicting or diagnosing early-, mid-, or late-pregnancy preeclampsia, including a composition for predicting or diagnosing the preeclampsia.

Another aspect is to provide a method of providing information for predicting or diagnosing early-, mid-, or late-pregnancy preeclampsia, the method including the steps of: measuring a level of a protein or a level of mRNA of a gene encoding the protein in a sample isolated from a subject in its early, mid, or late stage of pregnancy; and comparing the level of the protein or the level of mRNA of the gene encoding the protein to that in a sample of a normal control group in its early, mid, or late stage of pregnancy.

Another aspect is to provide a method of predicting or diagnosing early-, mid-, or late-pregnancy preeclampsia, the method including the steps of: measuring a level of a protein or a level of mRNA of a gene encoding the protein in a sample isolated from a subject in its early, mid, or late stage of pregnancy; and comparing the level of the protein or the level of mRNA of the gene encoding the protein to that in a sample of a normal control group in its early, mid, or late stage of pregnancy.

### Technical Solutions

One aspect provides a biomarker composition for predicting or diagnosing early-, mid-, or late-pregnancy preeclampsia, including a protein or a gene encoding the protein.

Another aspect provides a use for predicting or diagnosing early-, mid-, or late-pregnancy preeclampsia, of a biomarker including a protein or a gene encoding the protein.

Another aspect provides a composition for predicting or diagnosing early-, mid-, or late-pregnancy preeclampsia, including a formulation for measuring a level of a protein or a level of mRNA of a gene encoding the protein.

Another aspect provides a use for predicting or diagnosing early-, mid-, or late-pregnancy preeclampsia, of a formulation for measuring a level of a protein or a level of mRNA of a gene encoding the protein.

Another aspect provides a use for producing a composition for predicting or diagnosing early-, mid-, or late-pregnancy preeclampsia, of a formulation for measuring a level of a protein or a level of mRNA of a gene encoding the protein.

According to one embodiment, in a biomarker composition for predicting or diagnosing early-pregnancy preeclampsia or a composition for predicting or diagnosing early-pregnancy preeclampsia, the protein may include one or more proteins selected from the group consisting of APOE, MMP2, LTA4H, ENG, GPT, SERPINB3, KRT9, CHI3L1, DSC2, SPP2, GFUS, CD109, SCARA5, MYOC, ADA2, PSMD9, TREH, SERPINA7, TUBB, PFN1, HLA-C, G6PD, ANXA3, RAN, CTBS, GDF15, XYLT2, and GP6.

According to one embodiment, in a biomarker composition for predicting or diagnosing mid-pregnancy preeclampsia or a composition for predicting or diagnosing mid-pregnancy preeclampsia, the protein may include one or more proteins selected from the group consisting of IGLC2, IGLC3, IGLC6, IGLC7, PLXNA2, GBA, ALDOB, COL1A2, MMP9, EPHA1, PTPRB, GSTO1, AOC3, ERAP2, SEZ6L2, ADAMTSL4, SCARA5, VAT1, CPVL, PSG8, ARID1B, WIPF1, CP, AGRN, CES2, TREH, APOB, SERPINE2, ITGA5, F7, HLA-C, GHR, LIPC, CAST, PRCP, ADAM17, FLNC, NOTUM, and VWA1.

According to one embodiment, in a biomarker composition for diagnosing late-pregnancy preeclampsia or a composition for diagnosing late-pregnancy preeclampsia, the protein may include one or more proteins selected from the group consisting of GSK3A, PSMA7, SUSD5, CFD, COL3A1, FN1, FTL, CAT, SOD2, ALDOB, APP, CA3, DCN, MMP2, ITGA5, PAEP, ESD, COL6A2, F5, CEACAM1, GOT1, FLT1, SDC1, CAST, COL5A1, PEBP1, SDC4, HSPA4, CHI3L1, APLP1, FBLN2, TRAP1, SELENBP1, GFUS, POSTN, CSPG4, ADAMTSL4, CEMIP, KCTD12, COLEC11, PAPPA2, SEZ6L, CDH23, GKN1, CRYBB3, ADAM12, TREH, SEMA7A, CLN5, AGT, C9, LRG1, TTR, HPX, CLEC3B, SERPINA7, SERPIND1, C2, GUSB, CSF1, C4A, C4B, HLA-C, PTPRF, PSG2, LBP, FBLN1, CDH5, ENPEP, SOS1, PCDH1, DDR1, MGAT2, GALNT1, PTK7, DSG2, ADGRG6, ITIH5, OIT3, NRCAM, SEMA6A, DPEP2, CDHR5, IL1RAP, FETUB, PSG8, ST3GAL6, and PLXND1.

In this specification, the term “preeclampsia (PE)” refers to a hypertensive disease associated with pregnancy, and specifically may be a concept including gestational hypertension, and preeclampsia and eclampsia, which are more advanced forms of the condition. Symptoms of preeclampsia include headache, edema, nausea, vomiting, foamy urine, or the like.

In one embodiment, the condition of preeclampsia may be one or more selected from the group consisting of chronic hypertension, gestational hypertension, preeclampsia, eclampsia, or combined preeclampsia.

The early pregnancy refers to the period spanning from the start of the last menstrual period to 13 weeks and 6 days, the mid pregnancy refers to the period spanning from the start of 14 weeks to 27 weeks and 6 days, and the late pregnancy refers to the period spanning from the start of 28 weeks to 42 weeks.

In this specification, the term “diagnosis” may refer to determining an object's susceptibility to a specific disease or condition, determining whether an object has a specific disease or condition, determining the prognosis of an object having a specific disease or condition, or monitoring such prognosis.

Conventionally, it was possible to diagnose preeclampsia only after 20 weeks of pregnancy, but the composition for predicting or diagnosing preeclampsia or the method of providing information for predicting or diagnosing the same according to the present disclosure may enable effective prediction of preeclampsia in the early and middle stages of pregnancy and simplified diagnosis the middle and late stages of pregnancy.

In this specification, the terms “marker” or “biomarker” refer to a substance capable of distinguishing and evaluating individuals with a disease from individuals without the disease, and may include organic biomolecules such as polypeptides, proteins, nucleic acids (e.g., mRNA, etc.), lipids, glycolipids, glycoproteins, sugars (monosaccharides, disaccharides, oligosaccharides, etc.), metabolites, or the like, which show an increase or decrease in individuals with a disease compared to individuals without the disease.

In this specification, the term “level measurement” may include measuring the degree of expression or activity of the marker for preeclampsia in a biological sample to predict or diagnose preeclampsia.

Specifically, measuring a level of a protein may include measuring the degree of expression of the protein that changes with the onset of preeclampsia in pregnant women in the early, middle, or late stages of pregnancy.

Specifically, measuring a level of mRNA may include measuring the degree of expression of mRNA that changes with the onset of preeclampsia in pregnant women in the early, middle, or late stages of pregnancy.

In one embodiment, the formulation for measuring the level of the protein may include one or more selected from the group consisting of an antibody, interacting protein, oligopeptide, ligand, nanoparticle, and aptamer that specifically bind to the protein or a peptide fragment thereof.

The protein may include not only the protein itself, but also protein isoforms or protein variants that may be produced by splicing and variable promoters or produced due to genetic changes such as mutations or polymorphisms.

The antibody may refer to a substance that specifically binds to the protein and causes an antigen-antibody reaction. The antibody may include a polyclonal antibody, a monoclonal antibody, or a recombinant antibody. The antibody may be easily produced using technology widely known in the art. The antibody may be separated and purified using methods such as gel electrophoresis, dialysis, salt precipitation, ion exchange chromatography, and affinity chromatography. In addition, the antibody may include not only a complete form having two full-length light chains and two full-length heavy chains, but also functional fragments of the antibody molecule.

The interacting protein refers to a protein having a protein-protein interaction (PPI) with the protein, and may refer to a protein having a highly specific physical contact as a result of biochemical actions regulated by interactions including electrostatic force, hydrogen bonding, and hydrophobic effects.

The oligopeptide is composed of 2 to 20 amino acids as a peptide and may include, but is not limited to, dipeptides, tripeptides, tetrapeptides, and pentapeptides.

The aptamer refers to a single-stranded oligonucleotide and may refer to a nucleic acid molecule having binding activity to the protein. The aptamer may have various secondary or tertiary structures depending on its base sequence and may have high affinity for specific substances, such as in antigen-antibody reactions. The aptamer may be RNA, DNA, modified nucleic acid, or a mixture thereof, and may be in a linear or circular form.

In one embodiment, the formulation for measuring the level of mRNA of the gene may include one or more selected from the group consisting of a primer, a probe, a peptide nucleic acid (PNA), and an antisense nucleotide that specifically binds to the gene.

The primer is a fragment that recognizes a target gene sequence and may include a forward and reverse primer pair, and preferably, may be a primer pair that provides analysis results having specificity and sensitivity. For a primer, of which a nucleic acid sequence is inconsistent with a non-target sequence present in a sample, amplifying only the target gene sequence containing a complementary primer binding site and not inducing non-specific amplification, high specificity may be conferred.

The probe refers to a nucleic acid fragment, such as RNA or DNA, that may specifically bind to a target nucleic acid, for example, mRNA, and may be labeled to confirm the presence, content, and expression level of a specific m RNA.

The probe may be produced in the form of an oligonucleotide probe, a single-strand DNA probe, a double-strand DNA probe, an RNA probe, or the like. The conditions for selection of appropriate probes and hybridization may be appropriately selected according to techniques known in the related technical field.

The PNA refers to an artificially synthesized polymer similar to DNA or RNA. While DNA has a phosphate-ribose sugar backbone, PNA has a repeating N-(2-aminoethyl)-glycine backbone connected by peptide bonds, which may significantly increase the binding strength and stability to DNA or RNA of a target sequence.

The antisense may refer to an oligomer having a backbone between a nucleotide base sequence and a subunit, in which the antisense oligomer hybridizes with a target sequence within RNA by Watson-Crick base pairing, allowing the formation of mRNA and RNA: oligomer heterodimers within the target sequence. Oligomers may have exact or approximate sequence complementarity with respect to the target sequence.

Since the mRNA information of the protein or the gene encoding the protein according to the present disclosure is known in the art, those skilled in the art can design a formulation for measuring the level of the protein or the level of mRNA of the gene encoding the protein based thereon.

Another aspect provides a kit for predicting or diagnosing early-, mid-, or late-pregnancy preeclampsia, including a composition for predicting or diagnosing the preeclampsia.

In one embodiment, the kit may be, but is not limited to, an enzyme-linked immunosorbent assay (ELISA) kit, a protein chip kit, a rapid kit, a multiple reaction monitoring (MRM) kit, a reverse transcription polymerase chain reaction (RT-PCR) kit, or a deoxyribonucleic acid (DNA) chip kit.

The kit may further include one or more other component compositions, solutions, or devices suitable for the analysis method.

The ELISA kit may include essential elements necessary to perform an ELISA. The ELISA kit may include an antibody specific to the protein. The antibody, which has high specificity and affinity for a marker protein and has little cross-reactivity with other proteins, may include a monoclonal antibody, a polyclonal antibody, or a recombinant antibody. In addition, the ELISA kit may include an antibody specific to a control protein (e.g., a housekeeping protein). In addition, the ELISA kit may also include a reagent capable of detecting conjugated antibodies, for example, labeled secondary antibodies, chromophores, enzymes (e.g., conjugated with the antibody), and other substances capable of binding to its substrate or antibody.

The protein chip kit may include essential elements necessary to perform a protein chip operation. The protein chip kit may include a substrate to which a protein or a fragment thereof is attached, and a reagent, a formulation, an enzyme, or the like for producing a fluorescently labeled probe. The substrate may include a control protein or a fragment thereof, but is not limited thereto.

The rapid kit may consist of a sample pad to which a sample is applied, a membrane on which an antibody is immobilized, and an absorbent pad capable of absorbing the sample, but is not limited thereto.

The MRM kit may further include essential elements necessary to perform multiple reaction monitoring. The MRM kit may include a peptide that selectively recognizes the protein.

The RT-PCR kit may further include essential elements necessary to perform an RT-PCR. The RT-PCR kit may include a primer pair specific to the gene. The primer is a nucleotide having a sequence specific to the nucleic acid sequence of the gene, and may have a length of about 7 bp to 50 bp, more preferably about 10 bp to 30 bp. It may also include a primer specific to the nucleic acid sequence of a control gene (e.g., a housekeeping gene). The RT-PCR kit may also include, but is not limited to, a test tube or other suitable containers, a reaction buffer (with varying pH and magnesium concentrations), deoxynucleotides (dNTPs), an enzyme such as Taq-polymerase and reverse transcriptase, DNase, RNase inhibitors, DEPC-water, sterile water, or the like.

The DNA chip kit may include essential elements necessary to perform a DNA chip operation. The DNA chip kit may include a substrate to which cDNA or oligonucleotides corresponding to a gene or a fragment thereof are attached, and a reagent, a formulation, an enzyme, or the like for producing a fluorescently labeled probe. The substrate may include cDNA or oligonucleotides corresponding to a control gene (e.g., a housekeeping gene) or a fragment thereof, but is not limited thereto.

In addition, the kit may be an integrated magneto-electrochemical sensor kit. An integrated magneto-electrochemical sensor refers to a system that detects electrical signals through enzymatic signal amplification using magnetic beads. Specifically, it refers to a system that, after a substance capable of detecting a target substance (e.g., an antibody) is attached to a magnetic bead, detects electrical signals generated by enzymatic signal amplification by a chromogenic substance capable of detecting that target substance (e.g., horseradish peroxidase (HRP), alkaline phosphatase (ALP), α-D-galactosidase (α-Gal), etc.).

In one embodiment, the kit may include a formulation, a device, and a computer with a built-in algorithm for measuring the level of the protein or the level of mRNA of the gene encoding the protein, and may associate a result of measuring the level of the marker with the prediction or diagnosis of preeclampsia through the algorithm.

Another aspect provides a method of providing information for predicting or diagnosing early-, mid-, or late-pregnancy preeclampsia, the method including the steps of: measuring a level of a protein or a level of mRNA of a gene encoding the protein in a sample isolated from a subject in its early, mid, or late stage of pregnancy; and comparing the level of the protein or the level of mRNA of the gene encoding the protein to that in a sample of a normal control group in its early, mid, or late stage of pregnancy.

Another aspect provides a method of predicting or diagnosing early-, mid-, or late-pregnancy preeclampsia, the method including the steps of: measuring a level of a protein or a level of mRNA of a gene encoding the protein in a sample isolated from a subject in its early, mid, or late stage of pregnancy; and comparing the level of the protein or the level of mRNA of the gene encoding the protein to that in a sample of a normal control group in its early, mid, or late stage of pregnancy.

In this specification, the term “subject” refers to a mammal including a human, which may be selected from the group consisting of, for example, humans, rats, mice, guinea pigs, hamsters, rabbits, monkeys, dogs, cats, cattle, horses, pigs, sheep, and goats, and preferably may be a human, but is not limited thereto.

In this specification, the term “sample” means any substance, biological fluid, tissue, or cell obtained from or derived from a patient who has developed preeclampsia or a subject suspected of having developed preeclampsia, and may be one or more selected from the group consisting of, for example, whole blood, leukocytes, peripheral blood mononuclear cells, leukocyte soft layer, blood including plasma and serum, sputum, tears, mucus, nasal fluid, nasal aspirate, breath, urine, semen, saliva, peritoneal lavage fluid, pelvic fluid, cystic fluid, cerebrospinal fluid, amniotic fluid, glandular fluid, pancreatic fluid, lymph fluid, pleural fluid, nipple aspirate, bronchial aspirate, synovial fluid, joint aspirate, tracheal secretion, cells, cell extracts, or cerebrospinal fluid, but preferably may be a liquid biopsy, for example, blood, serum, or plasma.

The method of the present disclosure according to one embodiment involves applying an appropriate treatment (e.g., removal of abundant protein, peptide formation, etc.) to a sample according to a method known in the art to measure the level of a marker protein or mRNA of a gene encoding the same in a sample isolated from a subject, but is not limited thereto.

In a method of providing information for predicting or diagnosing early-pregnancy preeclampsia according to one embodiment, the protein may include one or more proteins selected from the group consisting of APOE, MMP2, LTA4H, ENG, GPT, SERPINB3, KRT9, CHI3L1, DSC2, SPP2, GFUS, CD109, SCARA5, MYOC, ADA2, PSMD9, TREH, SERPINA7, TUBB, PFN1, HLA-C, G6PD, ANXA3, RAN, CTBS, GDF15, XYLT2, and GP6.

In a method of providing information for predicting or diagnosing mid-pregnancy preeclampsia according to one embodiment, the protein may include one or more proteins selected from the group consisting of IGLC2, IGLC3, IGLC6, IGLC7, PLXNA2, GBA, ALDOB, COL1A2, MMP9, EPHA1, PTPRB, GSTO1, AOC3, ERAP2, SEZ6L2, ADAMTSL4, SCARA5, VAT1, CPVL, PSG8, ARID1B, WIPF1, CP, AGRN, CES2, TREH, APOB, SERPINE2, ITGA5, F7, HLA-C, GHR, LIPC, CAST, PRCP, ADAM17, FLNC, NOTUM, and VWA1.

In a method of providing information for diagnosing late-pregnancy preeclampsia according to one embodiment, the protein may include one or more proteins selected from the group consisting of GSK3A, PSMA7, SUSD5, CFD, COL3A1, FN1, FTL, CAT, SOD2, ALDOB, APP, CA3, DCN, MMP2, ITGA5, PAEP, ESD, COL6A2, F5, CEACAM1, GOT1, FLT1, SDC1, CAST, COL5A1, PEBP1, SDC4, HSPA4, CHI3L1, APLP1, FBLN2, TRAP1, SELENBP1, GFUS, POSTN, CSPG4, ADAMTSL4, CEMIP, KCTD12, COLEC11, PAPPA2, SEZ6L, CDH23, GKN1, CRYBB3, ADAM12, TREH, SEMA7A, CLN5, AGT, C9, LRG1, TTR, HPX, CLEC3B, SERPINA7, SERPIND1, C2, GUSB, CSF1, C4A, C4B, HLA-C, PTPRF, PSG2, LBP, FBLN1, CDH5, ENPEP, SOS1, PCDH1, DDR1, MGAT2, GALNT1, PTK7, DSG2, ADGRG6, ITIH5, OIT3, NRCAM, SEMA6A, DPEP2, CDHR5, IL1RAP, FETUB, PSG8, ST3GAL6, and PLXND1.

In one embodiment, the step of measuring the level of the protein may be performed by one or more selected from the group consisting of a protein mass spectrometry, protein chip analysis, immunoassay, ligand binding assay, matrix-assisted laser desorption/ionization time-of-flight (MALDI-TOF) mass spectrometry, surface-enhanced laser desorption/ionization time-of-flight (SELDI-TOF) mass spectrometry, radioimmunoassay (RIA), radial immunodiffusion (RID), Ouchterlony immunodiffusion, rocket immunoelectrophoresis (RIE), immunohistochemistry (IHC), complement fixation test (CFT), two-dimensional electrophoresis (2DE), liquid chromatography-mass spectrometry (LC-MS), liquid chromatography-mass spectrometry/mass spectrometry (LC-MS/MS), western blotting, and enzyme-linked immunosorbent assay (ELISA).

In one embodiment, the step of measuring the level of mRNA of the gene may be performed by one or more selected from the group consisting of a reverse transcription polymerase chain reaction (RT-PCR), competitive RT-PCR, real-time RT-PCR, RNase protection assay (RPA), northern blotting, and DNA chips.

In one embodiment, the method of providing information for predicting or diagnosing early-pregnancy preeclampsia may further include the step of determining preeclampsia when the level of one or more proteins selected from the group consisting of APOE, MMP2, LTA4H, ENG, GPT, SERPINB3, KRT9, CHI3L1, DSC2, SPP2, GFUS, CD109, SCARA5, MYOC, and ADA2, or the level of mRNA of a gene encoding the proteins is increased compared to that in a control group, or determining preeclampsia when the level of one or more proteins selected from the group consisting of PSMD9, TREH, SERPINA7, TUBB, PFN1, HLA-C, G6PD, ANXA3, RAN, CTBS, GDF15, XYLT2, and GP6, or the level of mRNA of a gene encoding the proteins is decreased compared to that in the control group.

In one embodiment, the method of providing information for predicting or diagnosing mid-pregnancy preeclampsia may further include the step of determining preeclampsia when the level of one or more proteins selected from the group consisting of IGLC2, IGLC3, IGLC6, IGLC7, PLXNA2, GBA, ALDOB, COL1A2, MMP9, EPHA1, PTPRB, GSTO1, AOC3, ERAP2, SEZ6L2, ADAMTSL4, SCARA5, VAT1, CPVL, and PSG8, or the level of mRNA of a gene encoding the proteins is increased compared to that in a control group, or determining preeclampsia when the level of one or more proteins selected from the group consisting of ARID1B, WIPF1, CP, AGRN, CES2, TREH, APOB, SERPINE2, ITGA5, F7, HLA-C, GHR, LIPC, CAST, PRCP, ADAM17, FLNC, NOTUM, and VWA1, or the level of mRNA of a gene encoding the proteins is decreased compared to that in the control group.

In one embodiment, the method of providing information for diagnosing late-pregnancy preeclampsia may further include the step of determining preeclampsia when the level of one or more proteins selected from the group consisting of GSK3A, PSMA7, SUSD5, CFD, COL3A1, FN1, FTL, CAT, SOD2, ALDOB, APP, CA3, DCN, MMP2, ITGA5, PAEP, ESD, COL6A2, F5, CEACAM1, GOT1, FLT1, SDC1, CAST, COL5A1, PEBP1, SDC4, HSPA4, CHI3L1, APLP1, FBLN2, TRAP1, SELENBP1, GFUS, POSTN, CSPG4, ADAMTSL4, CEMIP, KCTD12, COLEC11, PAPPA2, SEZ6L, CDH23, and GKN1, or the level of mRNA of a gene encoding the proteins is increased compared to that in a control group, or determining preeclampsia when the level of one or more proteins selected from the group consisting of CRYBB3, ADAM12, TREH, SEMA7A, CLN5, AGT, C9, LRG1, TTR, HPX, CLEC3B, SERPINA7, SERPIND1, C2, GUSB, CSF1, C4A, C4B, HLA-C, PTPRF, PSG2, LBP, FBLN1, CDH5, ENPEP, SOS1, PCDH1, DDR1, MGAT2, GALNT1, PTK7, DSG2, ADGRG6, ITIH5, OIT3, NRCAM, SEMA6A, DPEP2, CDHR5, IL1RAP, FETUB, PSG8, ST3GAL6, and PLXND1, or the level of mRNA of a gene encoding the proteins is decreased compared to that in the control group.

In this specification, the terms “increase in the level of a protein” or “increase in the level of mRNA of a gene” mean that the level of a protein or mRNA in a sample of a subject is increased significantly to a measurable degree compared to that in a control group, and may mean an increase by, for example, about 1.1 times or more, 1.1 to 2.5 times, 1.1 times, 1.2 times, 1.3 times, 1.4 times, 1.5 times, 1.6 times, 1.7 times, 1.8 times, 1.9 times, or 2 times or more.

In this specification, the terms “decrease in the level of a protein” or “decrease in the level of mRNA of a gene” mean that the level of a protein or mRNA in a sample of a subject is decreased significantly to a measurable degree compared to that in a control group, and may mean a decrease by, for example, about 0.9 times or less, 0.1 times to 0.9 times, 0.9 times, 0.8 times, 0.7 times, 0.6 times, 0.5 times, 0.4 times, 0.3 times, 0.2 times, or 0.1 times or less.

In one embodiment, the method of providing information for predicting or diagnosing preeclampsia may be performed in a form using a plurality of panels.

### Effects of Invention

According to an aspect, a composition for predicting or diagnosing preeclampsia or a method of providing information for predicting or diagnosing preeclampsia may simply and effectively predict or diagnose preeclampsia by measuring and comparing the level of a disease-specific protein that changes in a patient or the level of mRNA of a gene encoding the protein.

### BRIEF DESCRIPTION OF DRAWINGS

FIG. 1 shows the results of a heat map analysis of the level of an early-pregnancy preeclampsia-specific biomarker selected for predicting or diagnosing early-pregnancy preeclampsia.

FIG. 2 shows the results of a heat map analysis of the level of a mid-pregnancy preeclampsia-specific biomarker selected for predicting or diagnosing mid-pregnancy preeclampsia.

FIG. 3 shows the results of a heat map analysis of the level of a late-pregnancy preeclampsia-specific biomarker selected for diagnosing late-pregnancy preeclampsia.

### DETAILED DESCRIPTION OF THE INVENTION

Hereinafter, preferred embodiments are presented to aid in understanding the present disclosure. However, the following embodiments are provided merely to facilitate a better understanding of the present disclosure, and the content of the present disclosure is not limited by the following embodiments. However, the present disclosure is not limited to the embodiments described below but may be implemented in various forms.

Terms or words used in the specification and claims of the present disclosure shall not be interpreted as being limited to their ordinary or dictionary meanings, and shall be interpreted in a meaning and concept consistent with the technical spirit of the present disclosure, based on the principle that the inventor(s) may appropriately define the concept of the terms to best describe their invention.

Throughout the specification of the present disclosure, when a part is described as “comprising” a certain component, this means that, unless specifically stated otherwise, it does not exclude other components but may include additional components.

Throughout the specification of the present disclosure, “A and/or B” means A or B, or A and B.

### Example 1. Extraction of Plasma Proteins from Pregnant Mothers in their Early, Middle, and Late Pregnancy

Plasma proteins were extracted from early-, mid-, and late-pregnancy maternal plasma samples from 10 normal pregnant women and 10 pregnant women with preeclampsia.

### 1.1 Plasma Depletion

Each plasma sample was diluted 4-fold with buffer A, centrifuged at 16,000 g for 1 minute using a 0.22 μm spin filter, and protein samples were extracted by performing depletion using a Nexera HPLC system and a multi-affinity removal column (5188-6558) under the HPLC conditions shown in Table 1 below. Then, the protein samples were recovered using a fraction collector, and the recovered protein samples were vacuum-dried using SpeedVac.

TABLE 1

| Time | Module | Command | Value |
|---|---|---|---|
| 0.01 | Pumps | Flow rate | 0.125 |
| 0.33 | Valve | Close | — |
| 8.00 | Valve | Open | — |
| 17.00 | Valve | Close | — |
| 18.00 | Pumps | Flow rate | 0.125 |
| 18.01 | Pumps | Flow rate | 1 |
| 20.00 | Pumps | % B buffer | 0 |
| 20.01 | Pumps | % B buffer | 100 |
| 27.00 | Pumps | % B buffer | 100 |
| 27.01 | Pumps | % B buffer | 0 |
| 38.00 | Controller | Stop | — |

### 1.2 Protein Lysis and Digestion

Protein lysis and digestion are performed in the following steps (1) to (4).
(1) Protein lysis: 100 μl of lysis buffer (1% SDS in 50 mM TEAB) was added to the dried samples and sonicated for 4 minutes. The sonicated samples were vortexed and spun down, and then sonicated again for 4 minutes.
(2) Protein reduction and alkylation: A final concentration of 10 mM DTT (dithiothreitol) was added to each sample, placed in a Thermomixer, and reduced at 56° C. for 30 minutes. After the reaction was finished, a final concentration of 40 mM IAA (iodoacetamide) was added, and an alkylation reaction was performed at room temperature in a dark room for 30 minutes.
(3) S-TRAP protein digestion: A final concentration of 1.2% phosphoric acid solution was added to the alkylated samples, and a binding buffer (100 mM TEAB in 90% methanol) equivalent to 7 times the amount of lysis buffer was added. After placing the samples into an S-trap column and centrifuging at 1,000 g for 1 minute, the S-trap column was washed three times with 400 μl of binding buffer, and 0.4 μg/μl of Promega trypsin (sequencing grade) was added to 125 μl of digestion buffer (50 mM TEAB) at a ratio of 1:25 (=enzyme:protein), followed by incubation in a 37° C. incubator for 16 hours.
(4) Elusion & reconstitution: 80 μl of 50 mM TEAB was added to the S-trap column after enzyme digestion was completed, and the peptide samples were centrifuged at 1,000 g for 1 minute and transferred to a new tube. Then, 80 μl of 0.2% formic acid in water was added to the S-trap column, and the peptide samples were centrifuged at 1,000 g for 1 minute and collected. 80 μl of 0.2% formic acid in 50% ACN was added to the S-trap column, and the peptide samples collected after centrifugation at 1,000 g for 1 minute were vacuum-dried using SpeedVac. The dried samples were reconstituted in a 0.1% TFA solution, and the peptide concentration was measured using Nanodrop.

### Example 2. Selection of a Biomarker for Predicting or Diagnosing Early-Pregnancy Preeclampsia

To select a biomarker for predicting or diagnosing early-pregnancy preeclampsia, a protein extraction from maternal plasma in early pregnancy was performed according to Example 1, followed by an analysis using a mass spectrometer-based LC/MS.

Specifically, the mass spectrometric analysis was performed using a Nano LC-MS/MS (Thermo Dionex Ultimate 3000 coupled with Thermo Orbitrap Exploris 480) system according to the methods and conditions indicated in Table 2 below.

TABLE 2

| LC & LC-MS/MS | Nano-LC Ultimate 3000 & Thermo Orbitrap Exploris 480 | | |
|---|---|---|---|
| system | | | |
| Column | Trap column: Acclaim PepMap ™ 100, 100 μm × 2 cm | | |
| | Analytical column: PepMap ™ RSLC C18, 75 μm × 15 cm | | |
| Column Temp. | 50 °C | | |
| Flow rate | 1 μl/min | | |
| Injection volume | 2 μg | | |
| Mobile phase | A: 0.1% formic acid in water + 5% DMSO, | | |
| | B: 0.1% formic acid in 80% acetonitrile + 5% DMSO | | |
| | Time (min) | Solvent A (%) | Solvent B (%) |
| Gradient | 0 | 95 | 5 |
| | 5 | 85 | 15 |
| | 40 | 70 | 30 |
| | 46 | 60 | 40 |
| | 47 | 5 | 95 |
| | 51 | 5 | 95 |
| | 52 | 95 | 5 |
| | 60 | 95 | 5 |

| Ionization | ESI positive-ion mode | |
|---|---|---|
| Spray voltage | 2.5 kV | |
| Ion transfer | 275 °C | |
| tube temp. | | |
| MS1 resolution | 60,000 | |
| Scan range | 350-1,500 m/z | |
| FAIMS Voltages | On | |
| FAIMS | −35 CV | |
| Compensation | | |
| voltage | | |
| MS1 AGC target | 500% | |
| MS max IT | 20 ms | |
| MS2 resolution | 15,000 | |
| Precursor mass | 385-1,015 m/z | |
| range | | |
| Isolation window | 9 m/z | |
| Window overlap | 0 m/z | |
| FAIMS | −35 CV | −45 CV |
| Compensation | | |
| voltage | | |
| Loop control | 20 spectra | 20 spectra |

| | |
|---|---|
| MS2 AGC target | 300% |
| MS max IT | 25 ms |
| MS2 NCE | 28% |

For the LC-MS/MS data analysis, a data independent analysis (DIA) was performed on the results, and a database search was performed using DIA-NN, a software dedicated to the DIA data analysis.

The search parameters are as follows.
Library-free mode (in UniProt human protein database, peptide length 7-50, 1 or fewer missed cleavages, cysteine alkylation and methionine oxidation set to fixed and variable modification, respectively),
MBR (match-between-runs) mode.

Search results were filtered by precursor & protein-level FDR 1%. The median value was taken for the precursor to peptide to protein level summary, and the global distribution of all samples was adjusted by quantile normalization at each level to define the signature of a protein satisfying the following criteria (1) to (5) in order to identify blood proteins (i.e., signature) that are specifically overexpressed (up-regulated) in each condition (normal and preeclampsia):

Comparison of two groups,
(1) In each group, only proteins with missing value <=50% are retained,
(2) Within the top 15% of the empirical null fold-change distributions generated when log 2-fold-change randomly shuffles sample labels 1,000 times,
(3) empirical p-value <0.05,
(4) median expression in the condition of interest >0, and
(5) median expression in the other conditions <0.

As a result of performing, a protein exhibiting a final expression change was selected as a biomarker for predicting or diagnosing early-pregnancy preeclampsia.

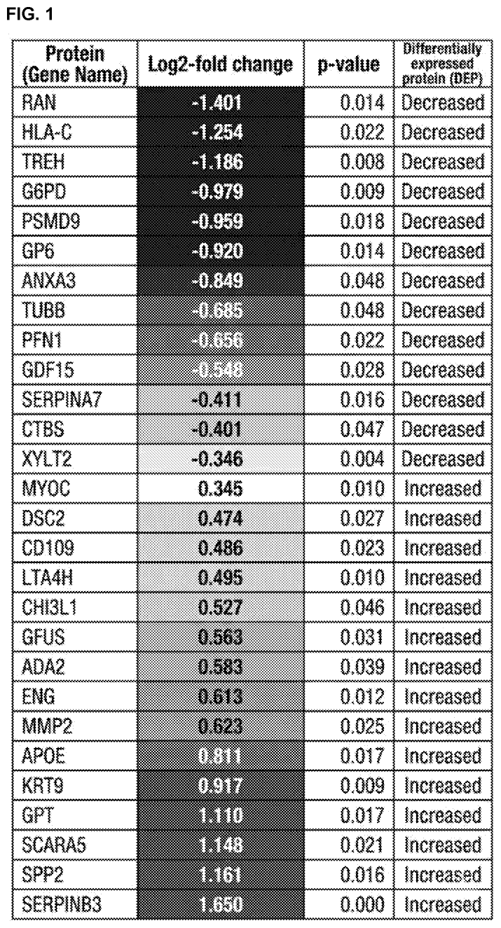

FIG. 1 shows the results of a heat map analysis of the level of an early-pregnancy preeclampsia-specific biomarker selected for predicting or diagnosing early-pregnancy preeclampsia.

As shown in FIG. 1, APOE, MMP2, LTA4H, ENG, GPT, SERPINB3, KRT9, CHI3L1, DSC2, SPP2, GFUS, CD109, SCARA5, MYOC, and ADA2 showed relatively high levels in a group with early-pregnancy preeclampsia compared to a control group, and
PSMD9, TREH, SERPINA7, TUBB, PFN1, HLA-C, G6PD, ANXA3, RAN, CTBS, GDF15, XYLT2, and GP6 showed relatively low levels in the group with early-pregnancy preeclampsia compared to the control group.

### Example 3. Selection of a Biomarker for Predicting or Diagnosing Mid-Pregnancy Preeclampsia

To select a biomarker for predicting or diagnosing mid-pregnancy preeclampsia, a protein extraction from maternal plasma in mid pregnancy was performed according to Example 1, followed by an analysis using a mass spectrometer-based LC/MS.

The protein analysis method was performed in the same manner as in Example 2 described above. As a result of performing, s protein exhibiting a final expression change was selected as a biomarker for predicting or diagnosing mid-pregnancy preeclampsia.

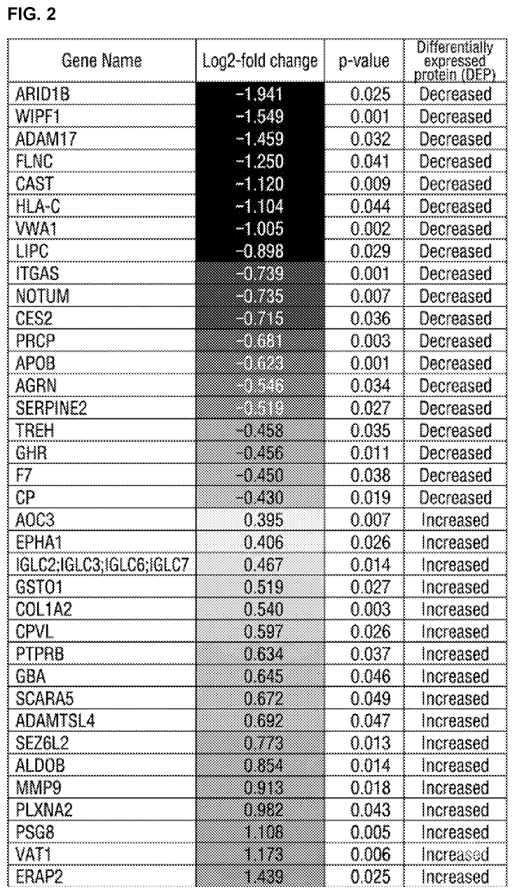

FIG. 2 shows the results of a heat map analysis of the level of mid-pregnancy preeclampsia-specific biomarker selected for predicting or diagnosing mid-pregnancy preeclampsia.

As shown in FIG. 2, IGLC2, IGLC3, IGLC6, IGLC7, PLXNA2, GBA, ALDOB, COL1A2, MMP9, EPHA1, PTPRB, GSTO1, AOC3, ERAP2, SEZ6L2, ADAMTSL4, SCARA5, VAT1, CPVL, and PSG8 showed relatively high levels in a group with mid-pregnancy preeclampsia compared to a control group, and
ARID1B, WIPF1, CP, AGRN, CES2, TREH, APOB, SERPINE2, ITGA5, F7, HLA-C, GHR, LIPC, CAST, PRCP, ADAM17, FLNC, NOTUM, and VWA1 showed relatively low levels in the group with mid-pregnancy preeclampsia compared to the control group.

### Example 4. Selection of a Biomarker for Diagnosing Late-Pregnancy Preeclampsia

To select a biomarker for diagnosing late-pregnancy preeclampsia, a protein extraction from maternal plasma in late pregnancy was performed according to Example 1, followed by an analysis using a mass spectrometer-based LC/MS.

The protein analysis method was performed in the same manner as in Example 2 described above. As a result of performing, a protein exhibiting a final expression change was selected as a biomarker for diagnosing late-pregnancy preeclampsia.

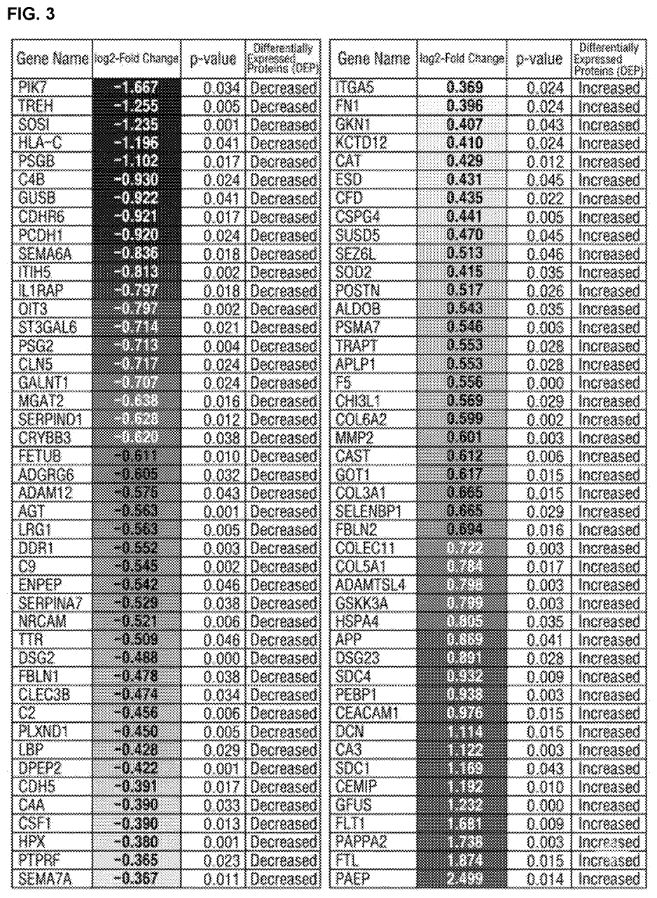

FIG. 3 shows the results of a heat map analysis of the level of late-pregnancy preeclampsia-specific biomarker selected for diagnosing late-pregnancy preeclampsia.

As shown in FIG. 3, GSK3A, PSMA7, SUSD5, CFD, COL3A1, FN1, FTL, CAT, SOD2, ALDOB, APP, CA3, DCN, MMP2, ITGA5, PAEP, ESD, COL6A2, F5, CEACAM1, GOT1, FLT1, SDC1, CAST, COL5A1, PEBP1, SDC4, HSPA4, CHI3L1, APLP1, FBLN2, TRAP1, SELENBP1, GFUS, POSTN, CSPG4, ADAMTSL4, CEMIP, KCTD12, COLEC11, PAPPA2, SEZ6L, CDH23, and GKN1 showed relatively high levels in a group with late-pregnancy preeclampsia compared to a control group, and
CRYBB3, ADAM12, TREH, SEMA7A, CLN5, AGT, C9, LRG1, TTR, HPX, CLEC3B, SERPINA7, SERPIND1, C2, GUSB, CSF1, C4A, C4B, HLA-C, PTPRF, PSG2, LBP, FBLN1, CDH5, ENPEP, SOS1, PCDH1, DDR1, MGAT2, GALNT1, PTK7, DSG2, ADGRG6, ITIH5, OIT3, NRCAM, SEMA6A, DPEP2, CDHR5, IL1RAP, FETUB, PSG8, ST3GAL6, and PLXND1 showed relatively low levels in the group with late-pregnancy preeclampsia compared to the control group.

From the foregoing description, those skilled in the art to which the present disclosure pertains will understand that the present disclosure may be implemented in other specific forms without altering its technical concept or essential features. In this regard, the experimental examples and embodiments described above should be understood as exemplary, not as restrictive, in all respects. The scope of the present disclosure should be interpreted as including all modifications or variations derived from the meaning and scope of the claims set forth below and equivalent concepts, rather than from the detailed description above.

### INDUSTRIAL APPLICABILITY

The present disclosure relates to a disease-specific biomarker for early prediction and diagnosis of preeclampsia and its use, which may be used for early prediction or diagnosis of preeclampsia.

### RELATED NATIONAL R&D PROJECTS

Project ID: 1465039723
Project Number: RS-2022-KH129953
Department Name: Ministry of Health and Welfare
Name of Project Management (Specialized) Agency: Korea Health Industry Development Institute
Research Project Name: Development of research-driven hospitals
Research Project Title: Establishment of a future innovation technology-based platform for resolving unmet medical needs in pregnancy and childbirth
Name of Project Implementing Organization: Bundang CHA Hospital
Research Period for the current year: 2023 Jan. 1-2023 Dec. 31

1. A method of providing information for predicting or diagnosing preeclampsia in an early stage of pregnancy, the method comprising: measuring a level of at least one protein selected from the group consisting of APOE, MMP2, LTA4H, ENG, GPT, SERPINB3, KRT9, CHI3L1, DSC2, SPP2, GFUS, CD109, SCARA5, MYOC, ADA2, PSMD9, TREH, SERPINA7, TUBB, PFN1, HLA-C, G6PD, ANXA3, RAN, CTBS, GDF15, XYLT2, and GP6, or a level of mRNA of a gene encoding the protein, in a sample isolated from a subject in its early pregnancy; and
comparing the level of the protein or the level of mRNA of the gene encoding the protein to that in a sample of a normal control group in its early stage of pregnancy.

2. The method of claim 1, wherein the measuring of the level of the protein is performed by at least one selected from the group consisting of a protein mass spectrometry, protein chip analysis, immunoassay, ligand binding assay, matrix-assisted laser desorption/ionization time-of-flight (MALDI-TOF) mass spectrometry, surface-enhanced laser desorption/ionization time-of-flight (SELDI-TOF) mass spectrometry, radioimmunoassay (RIA), radial immunodiffusion (RID), Ouchterlony immunodiffusion, rocket immunoelectrophoresis (RIE), immunohistochemistry (IHC), complement fixation test (CFT), two-dimensional electrophoresis (2DE), liquid chromatography-mass spectrometry (LC-MS), liquid chromatography-mass spectrometry/mass spectrometry (LC-MS/MS), western blotting, and enzyme-linked immunosorbent assay (ELISA).

3. The method of claim 1, wherein the measuring of the level of mRNA of the gene is performed by at least one selected from the group consisting of a reverse transcription polymerase chain reaction (RT-PCR), competitive RT-PCR, real-time RT-PCR, RNase protection assay (RPA), northern blotting, and DNA chips.

4. The method of claim 1, further comprising: when the level of the protein selected from the group consisting of APOE, MMP2, LTA4H, ENG, GPT, SERPINB3, KRT9, CHI3L1, DSC2, SPP2, GFUS, CD109, SCARA5, MYOC, and ADA2, or the level of mRNA of the gene encoding the protein is increased compared to that in the control group, determining preeclampsia; or
when the level of the protein selected from the group consisting of PSMD9, TREH, SERPINA7, TUBB, PFN1, HLA-C, G6PD, ANXA3, RAN, CTBS, GDF15, XYLT2, and GP6, or the level of mRNA of the gene encoding the protein is decreased compared to that in the control group, determining preeclampsia.

5. A composition for predicting or diagnosing preeclampsia in an early stage of pregnancy, comprising a formulation for measuring a level of at least one protein selected from the group consisting of APOE, MMP2, LTA4H, ENG, GPT, SERPINB3, KRT9, CHI3L1, DSC2, SPP2, GFUS, CD109, SCARA5, MYOC, ADA2, PSMD9, TREH, SERPINA7, TUBB, PFN1, HLA-C, G6PD, ANXA3, RAN, CTBS, GDF15, XYLT2, and GP6, or a level of mRNA of a gene encoding the protein.

6. The composition of claim 5, wherein the formulation for measuring the level of the protein comprises at least one selected from the group consisting of an antibody, interacting protein, oligopeptide, ligand, nanoparticle, and aptamer that specifically binds to the protein or a peptide fragment thereof.

7. The composition of claim 5, wherein the formulation for measuring the level of mRNA of the gene comprises at least one selected from the group consisting of a primer, a probe, a peptide nucleic acid (PNA), and an antisense nucleotide that specifically binds to the gene.

8. A kit for predicting or diagnosing preeclampsia in an early stage of pregnancy, comprising the composition of claim 5.

9. The kit of claim 8, being an enzyme-linked immunosorbent assay (ELISA) kit, a protein chip kit, a rapid kit, a multiple reaction monitoring (MRM) kit, a reverse transcription polymerase chain reaction (RT-PCR) kit, or a deoxyribonucleic acid (DNA) chip kit.
